(19) United States
(12) Patent Application Publication
(10) Pub. No.: US 2005/0267516 A1
(43) Pub. Date: Dec. 1, 2005

(54) Embolic protection device for the prevention of stroke

(76) Inventors: Farzad Soleimani, Stanford, CA (US); Vikram Mohan, Palo Alto, CA (US); Prashanth Ranganathan, Chester Hill (AU); Surag Mantri, Chatsworth, CA (US)

Correspondence Address: BELL & ASSOCIATES, 416 FUNSTON ST., SUITE 100, SAN FRANCISCO, CA, 94118, US

(21) Appl. No.: 11/143,507

(22) Filed: Jun. 1, 2005

Related U.S. Application Data
(60) Provisional application No. 60/575,692, filed on Jun. 1, 2004.

(51) Int. Cl. A61M 29/00

(52) U.S. Cl. 606/200

(57) ABSTRACT

The present invention relates to a device that can be used to prevent embolisms from accessing the brain and causing a stroke. The device can also prevent pulmonary embolism and to treat an aneurysm. The device provides a significant improvement to any similar devices currently in use.

The present application claims priority to U.S. Provisional Patent Application Ser. No. 60/575,692 entitled “Divert-X: an embolic protection device for the prevention of stroke”, filed Jun. 1, 2004, which is herein incorporated by reference in their entirety for all purposes.

### FIELD OF THE INVENTION

The invention is a device comprising a body and fin that can be inserted into a blood vessel, such as the aorta, of a subject at risk for cerebrovascular embolism or stroke. The invention also describes a method of surgery to prevent cerebrovascular embolism or stroke or recurrent stroke. The method comprises placing the embolic protection device in physical contact with arterial ostia that open from a blood vessel.

### BACKGROUND

Atrial fibrillation (AF), acute myocardial infarction, valvular heart disease, infective endocarditis, nonbacterial thrombotic endocarditis, and atrial myxoma are some of the cardiac causes of cerebrovascular emboli (Vahedi et al (2000) Curr. Treat. Options Neurol. 2(4): 305-318). AF is the most prevalent cardiac arrhythmia, characterized by rapid uncoordinated atrial impulses at a rate of 300-600 beats per minute. Patterns of AF are paroxysmal, persistent, or silent. Paroxysmal refers to an on-and-off pattern of AF, in persistent AF there are no off periods, and silent AF refers to patterns that are not felt or recognized by the patient. All these patterns carry a similar risk for stroke.

Pathophysiology of Atrial Fibrillation

Theories of the mechanism of AF involve two main processes: enhanced automaticity in one or several rapidly depolarizing foci, and reentry involving one or more circuits (Rensma et al. (1988) Circ. Res.62: 395-410). Rapidly firing atrial foci, located in one or several of the superior pulmonary veins, can initiate AF in susceptible patients (Jais et al. (1997) Circulation 95: 572-576; Haissaguerre et al. (1998) N. Engl. J. Med. 339: 659-666). Foci also occur in the right atrium, and infrequently in the superior vena cava or coronary sinus (Jais et al. (1997) supra; Haissaguerre et al. (1998) supra; and Chen et al. (1999) J. Cardiovasc. Electrophysiol. 10: 328-335). The focal origin appears to be more important in paroxysmal AF than in persistent AF. Ablation of such foci can be curative (Jais et al. (1997) supra).

The multiple-wavelet hypothesis, as a mechanism of reentrant AF, was advanced by Moe and colleagues, who proposed that the fractionation of the wave fronts, as they propagate through the atria results in self-perpetuating “daughter wavelets”. The number of wavelets present at any time depends on the refractory period, mass, and conduction velocity in different parts of the atria (Moe and Abildskov (1959) Am. Heart J. 58: 59-70).

Pathophysiology of Thromboembolism Due to AF

During AF, synchronous mechanical atrial activity is disturbed, resulting in homodynamic impairment. This can give rise to thrombus formation and embolism to the systemic circulation. Thrombi associated with AF arise most frequently in the left atrial appendage, where the blood flow is very low in velocity. An unproven theory is that the endothelium lining the left atrial appendage is itself thrombogenic and the homodynamic impairment and the very low blood flow increase the likelihood of thrombogenesis in this area. A thrombus can dislodge and travel through the common carotid artery and the internal carotid artery to reach the brain. As it interrupts the flow in any part of this pathway, it will lead to thromboembolism. Cerebrovascular emboli in AF patients most often manifest themselves as transient ischemic attacks or ischemic strokes.

One in every six strokes occurs in patients with AF. Half a year after a stroke, two out of three victims are either dead or severely disabled, and all stroke survivors are at risk to suffer recurrent strokes. (See Mattle (2003) Cerebrovascular Dis. 16(Suppl. 1): 3-8.) Stroke is a leading cause of morbidity and mortality in North America, to the extent that patients who have survived a stroke are ineligible to remain on an organ-transplant waiting list. The age-adjusted average lifetime cost of stroke was estimated to be $104,000 in 1990 (Taylor et al. (1996) Stroke 27(9): 1459-1466).

At present there exist several devices that can be used to treat or prevent cerebrovascular embolism or stroke. They include a device assigned to Mindguard Ltd. (claimed in U.S. Pat. Nos. 6,673,089 and 6,712,834) and EMBOL-X (claimed in U.S. Pat. No. 6,258). The Mindguard device is a tubular stent placed at the bifurcation of the common carotid artery and the internal and external carotid arteries. EMBOL-X is a diverter that shunts blood (and emboli contained therein) past the aortic arch. Many prior art references describe the placement of stents via catheters (see for example, U.S. Pat. No. 6,395,014 B1), and several other references describe other techniques to filter out emboli (see for example, U.S. Pat. Nos. 6,712,834 B2, 6,673,089 B1, and 6,258,120 B11).

Rapp et al. have reported that emboli that cause ischemic stroke are usually larger than 200 μm in diameter (Rapp et al. (2000) J. Vasc. Surg. 32: 68-76). Furthermore, the DIVERTER device, developed by Mindguard, has openings that divert embolic materials larger than 300 μm in diameter. This suggests that emboli that are at least 200 to 300 μm in diameter may not be obstructed by the DIVERTER and that other methods and devices need to be developed in order to significantly reduce the risk of stroke due to embolism.

There exists a need, therefore, for safe, alternative devices and methods for preventing stroke that are easy to place via a catheter within the lumen of the aortic arch.

### BRIEF DESCRIPTION OF THE INVENTION

The present invention provides an embolic protection device that is shaped and adapted for placement in a blood vessel of a human and that comprises materials that are porous to blood, blood cells, blood products, blood metabolites, cell metabolites, pharmaceutical compositions, and drugs, but that adsorb and/or block, impede, and/or hinder the passage of particulate emboli. In one embodiment, the device is an embolic protection device.

In a first embodiment, the embolic protection device comprises a body and a fin, the body adapted for placement within the lumen of a first blood vessel and the fin adapted for placement against the wall of the blood vessel and the opening of other blood vessels that emerge from the blood vessel. In one embodiment the first blood vessel is aortic arch, and the device is placed against the superior wall of the aortic arch and the opening of the blood vessels that arise from the superior portion of the aortic arch. In one embodiment, the other blood vessels are selected from the group consisting of the aorta, the brachiocephalic artery, the common carotid artery, and the subclavian artery. In a preferred embodiment the fin has apertures sized to obstruct selectively the passage of particulate emboli therethrough. In a preferred embodiment the body is a stent.

In the alternative, the embolic protection device is adapted for placement in the aorta and/or the abdominal aorta and the other blood vessels having ostia in the aorta and/or the abdominal aorta are, for example, the thoracic intercostals arteries, the celiac artery, the superior mesenteric artery, the renal arteries, the inferior mesenteric artery, the left common iliac artery, the right common iliac artery, the internal iliac arteries, the external iliac arteries, the femoral arteries, the pulmonary artery, and the heart chamber.

In one embodiment, the embolic protection device comprises a drug-eluting composition, the composition selected from the group consisting of silicones, polyurethanes, polyesters, vinyl homopolymers and copolymers, acrylate homopolymers and copolymers, polyethers and cellulosics. In one embodiment of the invention, the embolic protection device comprises a drug eluting or drug coated stent, which will help prevent restenosis following implantation of the stent. An example of a drug eluting stent and a method for making the same is disclosed in U.S. Pat. No. 6,206,915, which is hereby incorporated herein by reference. The stent and/or fin may be coated with a drug delivery compound or it may be partially made of a drug delivery compound. (See, for example, U.S. Pat. No. 6,716,242.)

In another embodiment, the fin comprises a composition that adsorbs particulate emboli. In a preferred embodiment, the composition is selected from the group consisting of anti-thrombosis drugs such as but not limited to, the group consisting of fibrin, poly(L-lactic acid), poly(lactide-co-glycolide), poly(hydroxybutyrate-co-valerate), heparin or heparin fragments, aspirin, coumadin, tissue plasminogen activator (tPA), urokinase (uPA), hirudin, and streptokinase, antiproliferatives (methotrexate, cisplatin, fluorouracil, adriamycin, and the like) antioxidants (ascorbic acid, carotene, vitamin B, vitamin E, and the like), antimetabolites, thromboxane inhibitors, non-steroidal and steroidal anti-inflammatory drugs, β blockers, calcium channel blockers, genetic materials including DNA and RNA fragments, and complete expression genes, carbohydrates, and proteins including but not limited to antibodies (monoclonal and polyclonal), lymphokines and growth factors, prostaglandins, leukotrienes, clopidogrel, dypiramidol, beraprost sodium, (2-acetyloxy-4-trifluoromethyl)benzoic acid; anti-restenosis drugs such aspaclitaxel and rapamycin, everolimus, a cytostatic antiproliferative drug selected from the group including, but not limited to, sirolimus, anti-sense to c-myc, tacrolimus, everolimus, CC1-779, 7-epi-rapamycin, 7-thiomethyl-rapamycin, 7-epi-trimethoxyphenyl—rapamycin, 7-epi-thiomethyl-rapamycin, 7-demethoxy-rapamycin, 32-demethoxy-rapamycin and 2-desmethyl-rapamycin; anti-proliferative drugs and compounds, such as growth and differentiation modulators, such as, but not limited to, TGF-β and/or bone morphogenic protein(s) (BMPs); bFGF; IGF-I; IGF-II; or ascorbic acid.

In one further embodiment, the body comprises a material that is selected from the group consisting of stainless steel, copper, gold, platinum, silver, titanium, nickel-titanium alloy (such as, but not limited to, NITINOL), epoxy, polymers, and the like. In another embodiment of the invention, the body comprises a swellable polymeric material, the swellable polymeric material comprising a swelling gel that swells and expands in volume when it comes into fluid contact with water or the like. Preferably the swelling gel is a super absorbent polymer.

In another embodiment, the body also comprises bioactive materials such as fibronectin, laminin, elastin, collagen, and intergrins.

The fin comprises materials including, but not limited to, nickel-titanium alloy (such as, but not limited to, NITINOL), polymers such as silicone, polyurethane, polyethylene, acrylonitrile butadiene styrene (ABS), polycarbonate, polypropylene, styrene, polyamide (nylon), polyimide, poly-(ether block amide) (PEBAX), polyester, poly(vinyl) chloride (PVC), fluoropolymers (TEFLON), co-polymers, high-density polyethylene (HDPE), low-density polyethylene (LDPE), polytetrafluoroethylene (PTFE), polyetheretherketone (PEEK), or the like. In a yet further embodiment, the fin comprises an elastomeric material, the elastomeric material selected from the group consisting of polyurethane, poly(vinyl)chloride (PVC), silicone rubber, latex, and the like.

In one embodiment, the fin comprises a plurality of fine wires, the fine wires having a diameter of not greater than 100 μm. In one preferred embodiment the diameter is not greater than 66 μm. In another preferred embodiment the diameter is not greater than 50 μm. In a more preferred embodiment the diameter is not greater than 25 μm. Reinforcement elements such as metallic (for example, stainless steel, NITINOL, or chromel) or polymeric braids or coils can be used in construction of the fin and/or the body.

In another embodiment, the embolic protection device comprises elements to make the body and/or the fin more visible to x-ray imaging. These elements can include tantalum, platinum, iridium, gold, stainless steel, silver, nickel-titanium alloys, polymer compounding agents such as barium sulfate and titanium oxide.

The embolic protection device can further comprise a marker. The marker can be, but is not limited to, a dye, a radio-opaque material, a magnet, an ion source, or the like.

In another embodiment, the fin further comprises at least one aperture. In an alternative embodiment, the fin comprises a plurality of apertures.

In another embodiment, the embolic protection device further comprises a deploying catheter, the deploying catheter adapted for placement in a blood vessel, and the deploying catheter further adapted for positioning the device in the lumen of a blood vessel.

In a yet a further embodiment, the embolic protection device comprises a plurality of fins.

In a still further embodiment, the embolic protection device comprises a plurality of stents.

The invention also contemplates a method of using an embolic protection device to prevent a cerebrovascular embolism in an individual. The method comprises the steps of i) providing an individual at risk for having a cerebrovascular embolism (stroke), ii) providing an embolic protection device, the embolic protection device comprising a stent and a fin, and a deploying catheter comprising a lumen, the stent and fin being foldedly disposed in the lumen of the deploying catheter iii) inserting a catheter having a lumen into a blood vessel of the individual, iv) advancing the distal end of the catheter to a desired position in the blood vessel, the desired position being proximal to an opening of another blood vessel, v) inserting a guide-wire through the lumen of the catheter, vi) advancing the guide-wire so that the distal end of the guide-wire is positioned proximal to the distal end of the catheter, vii) withdrawing the catheter, viii) advancing the deploying catheter comprising the folded embolic protection device over the guide-wire so that the distal end of the deploying catheter is proximal to the distal end of the guide-wire, ix) deploying the stent and fin from the deploying catheter so that the stent is opened and positioned against the wall of the blood vessel and the fin is positioned against the opening of the other blood vessel, x) withdrawing the catheter from the blood vessel, and xi) leaving the stent and fin in place for a desired time period.

The invention also contemplates a method of using an embolic protection device to treat an individual having had a stroke to prevent recurrent stroke. The method comprises the steps of i) providing an individual having had a cerebrovascular embolism (stroke), ii) providing an embolic protection device, the embolic protection device comprising a stent and a fin, and a deploying catheter comprising a lumen, the stent and fin being foldedly disposed in the lumen of the deploying catheter iii) inserting a catheter having a lumen into a blood vessel of the individual, iv) advancing the distal end of the catheter to a desired position in the blood vessel, the desired position being proximal to an opening of another blood vessel, v) inserting a guide-wire through the lumen of the catheter, vi) advancing the guide-wire so that the distal end of the guide-wire is positioned proximal to the distal end of the catheter, vii) withdrawing the catheter, viii) advancing the deploying catheter comprising the folded embolic protection device over the guide-wire so that the distal end of the deploying catheter is proximal to the distal end of the guide-wire, ix) deploying the stent and fin from the deploying catheter so that the stent is opened and positioned against the wall of the blood vessel and the fin is positioned against the opening of the other blood vessel, x) withdrawing the catheter from the blood vessel, and xi) leaving the stent and fin in place for a desired time period.

The invention also contemplates a method of using an embolic protection device to treat an individual having an aneurysm. The method comprises the steps of i) providing an individual having an aneurysm, ii) providing an embolic protection device, the embolic protection device comprising a stent and a fin, and a deploying catheter comprising a lumen, the stent and fin being foldedly disposed in the lumen of the deploying catheter iii) inserting a catheter having a lumen into a blood vessel of the individual, iv) advancing the distal end of the catheter to a desired position in the blood vessel, the desired position being proximal to the aneurysm, v) inserting a guide-wire through the lumen of the catheter, vi) advancing the guide-wire so that the distal end of the guide-wire is positioned proximal to the distal end of the catheter, vii) withdrawing the catheter, viii) advancing the deploying catheter comprising the folded embolic protection device over the guide-wire so that the distal end of the deploying catheter is proximal to the distal end of the guide-wire, ix) deploying the stent and fin from the deploying catheter so that the stent is opened and positioned against the wall of the blood vessel and the fin is positioned against the aneurysm, x) withdrawing the catheter from the blood vessel, and xi) leaving the stent and fin in place for a desired time period.

The invention also contemplates a method of using an embolic protection device to prevent a pulmonary embolism in an individual. The method comprises the steps of i) providing an individual at risk for having a pulmonary embolism, ii) providing an embolic protection device, the embolic protection device comprising a stent and a fin, and a deploying catheter comprising a lumen, the stent and fin being foldedly disposed in the lumen of the deploying catheter iii) inserting a catheter having a lumen into a blood vessel of the individual, iv) advancing the distal end of the catheter to a desired position in the blood vessel, the desired position being proximal to an opening of another blood vessel, v) inserting a guide-wire through the lumen of the catheter, vi) advancing the guide-wire so that the distal end of the guide-wire is positioned proximal to the distal end of the catheter, vii) withdrawing the catheter, viii) advancing the deploying catheter comprising the folded embolic protection device over the guide-wire so that the distal end of the deploying catheter is proximal to the distal end of the guide-wire, ix) deploying the stent and fin from the deploying catheter so that the stent is opened and positioned against the wall of the blood vessel and the fin is positioned against the opening of the other blood vessel, x) withdrawing the catheter from the blood vessel, and xi) leaving the stent and fin in place for a desired time period.

In one embodiment the embolic protection device is attached and/or secured against or to the wall using an inflatable cuff, a balloon, a stylet, a hook, a clip, a staple, a coil, a barb, an adhesive, a serrated blade or knife, a threaded screw, a vacuum device, or the like.

### BRIEF DESCRIPTION OF THE DRAWINGS

FIG. 1 shows a three-quarter view of an embodiment of the embolic protection device.

FIG. 2 shows a three-quarter view of an alternative embodiment of the embolic protection device.

FIG. 3 illustrates a detail of the structure of the fin (3) illustrating the plurality of longitudinal parallel fine wires (5) and the supporting cross-wires (6).

FIG. 4 shows a three-quarter view of an alternative embodiment of the embolic protection device.

FIG. 5 shows a three-quarter view of an alternative embodiment of the embolic protection device.

FIG. 6 illustrates the blood vessels of the upper thorax, the neck, and head.

FIG. 7 illustrates an exemplary use of the embolic protection device in the aortic arch.

FIG. 8 illustrates an exemplary use of means for using the embolic protection device in the aortic arch.

FIG. 9 illustrates an exemplary means for positioning and/or placing the embolic protection device in an experimental model of the aortic arch and blood vessel openings.

FIG. 10 illustrates a detail of the experimental model of the aortic arch and blood vessel openings.

FIG. 11 illustrates the embolic protection device being used to isolate an aneurysm.

FIG. 12 illustrates the blood vessels of the abdomen.

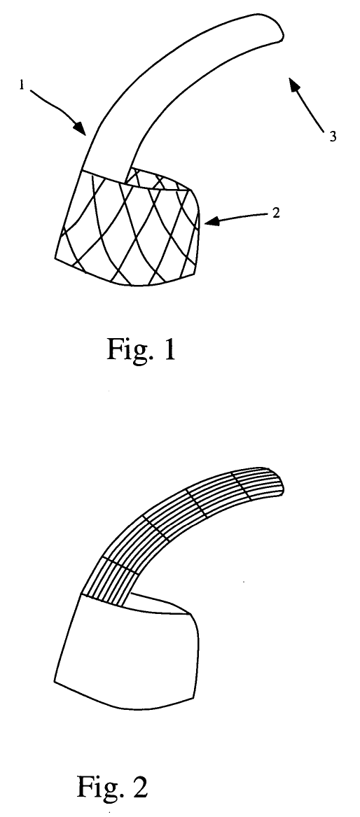

### DETAILED DESCRIPTION OF THE INVENTION

The embodiments disclosed in this document are illustrative and exemplary and are not meant to limit the invention. Other embodiments can be utilized and structural changes can be made without departing from the scope of the claims of the present invention.

As used herein and in the appended claims, the singular forms “a,” “an,” and “the” include plural reference unless the context clearly dictates otherwise. Thus, for example, a reference to “a fin” includes a plurality of such bearings, and a reference to “an aperture” is a reference to one or more apertures and equivalents thereof, and so forth.

The invention is a device adapted for placement in a blood vessel of a mammal and the device diverts embolisms from accessing another blood vessel. One preferable blood vessel is the aortic arch. The embolic protection device is placed in the aortic arch of a mammal to divert particulate embolisms from accessing any of the blood vessels opening from the aorta and that feed to regions of the brain. Diverting particulate embolisms from the brain can significantly reduce the likelihood of cerebrovascular embolism or stroke. Preferably the mammal is a human individual having had a stroke. More preferably the individual is an individual having had a stroke and at increased risk for another stroke.

The invention can be used together as a plurality of devices, each device being shaped and adapted for placement within a different region or portion of a blood vessel and/or the anatomy of an individual. For example, in some instances, an embolic protection device can be placed in the aortic arch, another placed in the abdominal aorta against the ostium of a renal artery, and/or another placed against the ostium of the celiac artery. In another example, in an individual at risk for DVT, the embolic protein device can be placed so that emboli are deflected and/or obstructed from entering the lung vasculature. Any combination of positions within the vasculature of an individual are possible and can be selected for by the physician and/or operator depending upon the clinical needs of an individual.

A specific embodiment of the embolic protection device (1) of the invention has the following features: a body (2) and a fin (3). The body can be a stent or another structure that is shaped and adapted for placement in a blood vessel. The embolic protection device can further comprise a releasable attaching means (4) disposed between the fin and the body as illustrated in FIG. 5 providing means to enable an operator to detach the fin from the body when required. In some cases it may be advantageous to remove the fin during surgery and replace the fin at a later time. The stent is made from a memory-metal alloy, such as NiTi alloy (for example, NITINOL), that is confined as a straightened shape in a lumen within the deploying catheter. The stent is deployed in the lumen of the aorta and, upon attaining the final shape, it is constrained against the wall of the aorta thereby securing the device in place.

The body can also comprise an inflatable cuff or balloon (FIG. 4, reference number 8), the cuff or balloon being shaped and adapted for placement and securing the body against the walls of the blood vessel. The cuff or balloon is fixed to the outer surface of the body. The cuff or balloon is shaped and sized in such a way that when positioned within the blood vessel and inflated, the cuff or balloon projects radially from the body, and impinges upon the inner walls of the blood vessel, thereby positioning and/or fixing the body in close proximity to the ostium of a blood vessel.

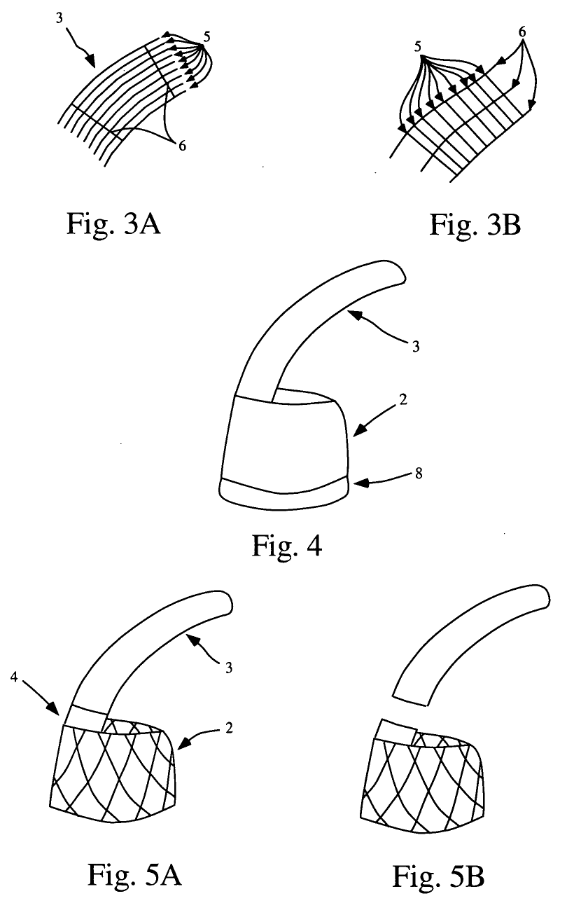

The fin (3) can comprise a plurality of fine wires (5), parallely extending along the longitudinal axis of the fin, the fine wires (5) being spaced apart from one another at a distance of not more than about 300 μm, thereby obstructing the passage of particulate emboli therethrough. Preferably, the spacing distance is not more than about 200 μm. More preferably, the spacing distance is about 100 μm. Alternatively, the spacing distance is about 50 μm. The spacing of the fine wires preferentially obstructs any particle with a minimum diameter of about 200 μm. Blood cells usually have a maximum diameter of about 7 to 20 μm and therefore are not obstructed or impeded from passing between the wires. To maintain the spacing distance, the fin can further have supporting cross wires (6) positioned perpendicular to the plurality of parallel wires being spaced at about 10 cm intervals on the fin. Alternatively, the cross-wires can be spaced at about 0.1 cm, 0.2 cm, 0.3 cm, 0.4 cm, 0.5 cm, 0.6 cm, 0.7 cm, 0.8 cm, 0.9 cm, 1.1 cm, 1.3 cm, 1.5 cm, 2.0,2.5 cm, 3 cm, 4 cm, 5 cm, 6 cm, 7 cm, 8 cm, 9 cm, and/or 10 cm intervals on the fin (See FIG. 3.)

The fine wires can have a diameter of not greater than 100 μm. Alternatively, the fine wires can have a diameter of not greater than 66 μm, 50 μm, 25 μm, 10 μm, 5 μm, 2 μm, or 1 μm.

In an alternative arrangement of the fine wires of the fin, the fine wires parallely extend perpendicular to the axis of the fin and further comprising at least two supporting cross wires to maintain the spacing distance, the supporting cross wires being spaced at about 1.5 cm intervals. Alternatively, the cross-wires can be spaced at about 0.05 cm, 0.1 cm, 0.15 cm, 0.2 cm, 0.25 cm, 0.3 cm, 0.35 cm, 0.4 cm, 0.45 cm, 0.5 cm, 0.55 cm, 0.6 cm, 0.65 cm, 0.7 cm, 0.75 cm, 0.8, 0.85 cm, 0.9, 0.95 cm, and/or 1.0 cm intervals on the fin. In addition, a plurality of cross-wires can be used to maintain the spacing between the fine wires and/or support the fine wires.

The invention also contemplates an arrangement of the wires wherein the cross-wires are at an angle to the longitudinal axis of the fin and the fine wires are at a different angle relative to the longitudinal axis of the fin.

The wires can be selected from the group consisting of a material including, but not limited to, nickel-titanium alloy (such as, but not limited to, NITINOL), polymers such as, but not limited to, silicone, polyurethane, polyethylene, acrylonitrile butadiene styrene (ABS), polycarbonate, polypropylene, styrene, polyamide (nylon), polyimide, poly-(ether block amide) (PEBAX), polyester, poly(vinyl) chloride (PVC), fluoropolymers (TEFLON), co-polymers, high-density polyethylene (HDPE), low-density polyethylene (LDPE), polytetrafluoroethylene (PTFE), polyetheretherketone (PEEK), or the like.

Alternatively, the fin is a semi-permeable or dialysis membrane, comprising apertures of size that only small compounds can pass through the fin. Such small compounds include, but are not limited to, blood metabolites, antibodies, proteins, acids, bases, salts (including organic salts), amino acids, sugars, lipids, peptides, lipoproteins, nucleic acids, synthetic biomolecules, pharmaceutical compositions, dissolved gasses, combinations thereof, and the like. Semi-permeable or dialysis membranes are well known to those of skill in the art and can be obtained, for example, from Sartorius (Gottingen, Germany), Whatman Inc. (Florham Park N.J.), Corning (Corning N.Y.) and Solvay Advanced Polymers (Alpharetta Ga.). The apertures can be sized to exclude particles and/or molecules of a particular size range, and in some cases, be entirely impermeable to molecules in the blood.

The body or stent can further comprise an attaching means, such as an inflatable cuff or balloon (6) having an inflation lumen that, when inflated, constrains the body or stent against the wall of the blood vessel, thereby preventing and/or reducing the likelihood of drift through the lumen of the blood vessel that may result from the high fluid flow rates and pressures associated with the pumping heart. The cuff or balloon may be inflated using fluids or gases well known in the art, such as, but not limited to, air, nitrogen, helium, water, saline, and the like. An inflation catheter can optionally be in fluid communication with the inflation lumen of the cuff or balloon. Alternatively, the cuff or balloon can be inflated in situ, the lumen of the cuff or balloon comprising a swelling material, preferably a biocompatible gel, that swells upon contact with a fluid, the wall of the cuff or balloon being permeable to a fluid, such as, but not limited to, blood or saline.

The fin can be constructed from a variety of materials including nickel-titanium alloy (such as, but not limited to, NITINOL), polymers such as, but not limited to, silicone, polyurethane, polyethylene, acrylonitrile butadiene styrene (ABS), polycarbonate, polypropylene, styrene, polyamide (nylon), polyimide, poly-(ether block amide) (PEBAX), polyester, poly(vinyl) chloride (PVC), fluoropolymers (TEFLON), co-polymers, high-density polyethylene (HDPE), low-density polyethylene (LDPE), polytetrafluoroethylene (PTFE), polyetheretherketone (PEEK), or the like. In a yet further embodiment, the fin comprises an elastomeric material, the elastomeric material selected from the group consisting of polyurethane, poly(vinyl)chloride (PVC), silicone rubber, latex, and the like.

In addition, the embolic protection device can comprise a drug eluting or drug coated stent, including drugs that will help prevent restenosis following implantation of the stent. An example of a drug eluting stent and a method for making the same is disclosed in U.S. Pat. No. 6,206,915, which is hereby incorporated herein by reference. The stent and/or fin may be coated with a drug delivery compound or it may be partially made of a drug delivery compound. The stent and/or fin is placed such that it delivers a sustained release of a drug. There are a number of viable pharmacologic therapies available. For example, drugs that predominantly affect slow pathway conduction include digitalis, calcium channel blockers, and beta-blockers. Drugs that predominantly prolong refractoriness, or time before a heart cell can be activated, produce conduction block in either the fast pathway or in accessory atroventricular (AV) connections including the class IA antiarrhythmic agents (quinidine, procainimide, and disopyrimide) or class IC drugs (flecainide and propafenone). The class III antiarrhythmic agents (sotolol or amiodorone) prolong refractoriness and delay or block conduction over fast or slow pathways as well as in accessory AV connections. Temporary blockade of slow pathway conduction is usually achieved by intravenous administration of adenosine or verapamil (Scheinman and Melvin (1994) Clin. Cardiol. 17:Supp. II-11-II-15). Other agents such as encainide, diltiazem, and nickel chloride are also available. (See, for example, U.S. Pat. No. 6,716,242.)

The device (1) may have a diameter of, for example, from about 3 mm to about 65 mm, or from about 3 mm to about 45 mm, or for example about 5 mm, 7 mm, 12 mm, 25 mm, 18 mm, 22 mm, 25 mm, 28 mm, 31 mm, 35 mm, 40 mm, 45 mm, 50 mm, 55 mm, or 60 mm. The length of the device may be any length compatible with its function of placing an embolic protection device or other medical device within the aortic arch, and the device may (or may not) be shorter than the deploying device and/or system that is used to deploy it into the aorta. For example, the device may be from about 0.5 cm to about 10 cm in length, or for example about 1 cm, 1.5 cm, 2 cm, 2.5 cm, 3 cm, 3.5 cm, 4 cm, 5 cm, 6 cm, 7 cm, 8 cm, 9 cm, or 10 cm in length. The body of the invention may be of variable fixed lengths, or it may be of dynamically adjustable length by use of a telescoping designs. The body of the invention is generally a short cylinder, though it may be of any suitable cross-sectional shape such as oval or polygonal. Preferably the fin has a longitudinal axial length greater than the longitudinal axial length of the body or stent. The body of the invention may be rigid or may be flexible. A flexible body is desirable when using a flexible catheter.

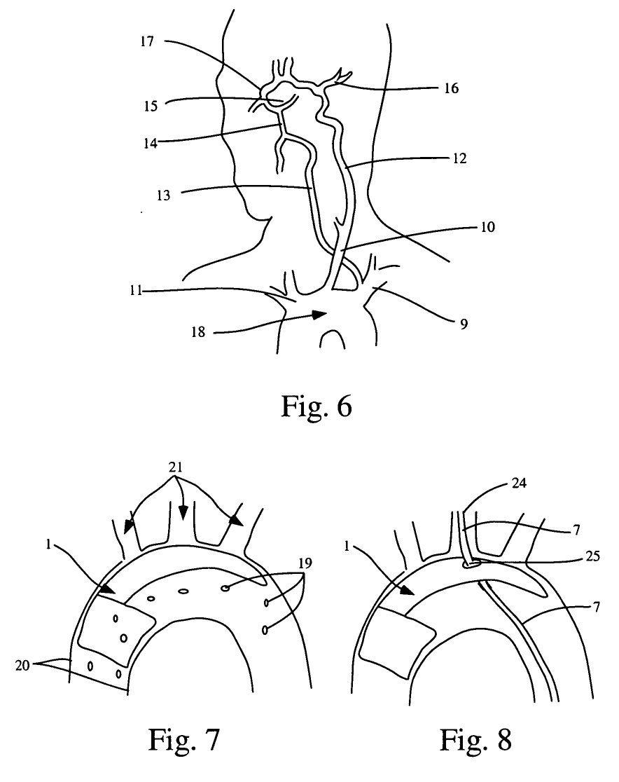

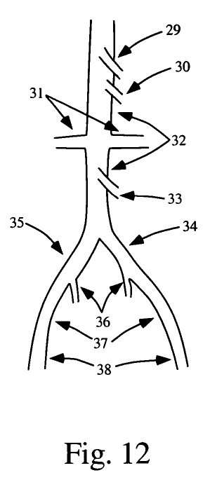

The embolic protection device can be shaped and adapted for placement in any blood vessel of the individual. The fin is shaped and adapted for placement against an ostium. The blood vessels that originate from the aortic arch (18) and that conduct blood to the upper thorax, the upper extremities, the neck, and the head are illustrated on FIG. 6 and comprise the following: the subclavian artery (9), the common carotid (10), the brachiocephalic artery (11), the internal carotid artery (12), the vertebral artery (13), the basilar artery (14), the posterior cerebral artery (15), the middle cerebral artery (16), and the anterior cerebral artery (17). The blood vessels that originate from the aorta and that conduct blood to the lower thorax are the thoracic intercostal arteries. The blood vessels that originate from the abdominal aorta (32) and that conduct blood to the abdomen and the lower extremities are illustrated on FIG. 12 and comprise the following: the celiac artery (29), the superior mesenteric artery (30), the renal arteries (31), the inferior mesenteric artery (33), the left common iliac artery (34), the right common iliac artery (35), the internal iliac arteries (36), the external iliac arteries (37), and the femoral arteries (38).

The embolic protection device is placed in a blood vessel, such as for example, the aortic arch, the stent is opened and placed in position such that the mesh components of the stent are against the inner wall of the aorta (20) and the fin is positioned adjacent to the openings of the three arterial openings in the aortic arch (21). The fin diverts any particulate emboli (19) from passing though the fin and into the blood vessels (21).

Methods for loading stents into the lumen of a blood vessel or other anatomical structure using a catheter and guide-wire or the like are well known to those of skill in the art and are disclosed in, for example, U.S. Pat. No. 6,859,986 to Jackson et al., issued Mar. 1, 2005; U.S. Pat. No. 6,811,560 to Jones et al., issued Nov. 2, 2004; U.S. Pat. No. 6,818,013 to Mitelberg et al., issued Nov. 16, 2004; U.S. Pat. No. 6,146,358 to Rowe, issued Nov. 14, 2000; and U.S. Pat. No. 4,913,141 to Hillstead, issued Apr. 3, 1990. Such a catheter for deploying the body and fin can be a deploying catheter, the body and fin folded in such a way as to be constrained within the lumen of the deploying catheter. When the body and fin are deployed from the deploying catheter they resume a shape that the body and/or fin have shaped and adapted for placement in the blood vessel.

The invention contemplates that any number of the embolic protection devices disclosed herein may be placed in the vasculature or other anatomical structure of an individual. In one example, the embolic protection device may be placed in the abdominal aorta whereby the fin prevents and/or obstructs emboli from accessing a renal artery. In another example, the embolic protection device may be placed at the bifurcation of common carotid artery, the fin covering the ostium of internal carotid artery in order to divert emboli to the external carotid artery.

Once in place in the lumen of the blood vessel, the embolic protection device can also be used for placing a catheter (7) in at least one of the three arterial openings in the aortic arch, as illustrated in FIG. 8. The catheter can comprise a marker at the distal end (24). The marker can be, but is not limited to, a dye, a radio-opaque material, a magnet, an ion source, or the like. In the alternative, the catheter can be visualized using other methods well know to those of skill in the art, such as, but not limited to, intravascular ultrasound or the like.

The catheter is advanced through the vasculature and the distal end (24) is inserted through one of the apertures defined by the parallel fine wires and the cross-wires in the fin. The catheter is then advanced longitudinally through the aperture (25) of the fin and into a preferred arterial opening or ostium. The catheter can be used to sample body fluids in the upper thorax, the neck, and/or the head, and thereby determine the presence of embolisms or other particulates. The catheter can also comprise a sensor to detect changes in blood chemistry, blood metabolites, and the like. The catheter can also comprise a drug delivery device to deliver drugs at a target in the upper thorax, the neck, and/or the head. Such drugs can include, but are not limited to, chemotherapeutic agents, anticoagulants, hormones, growth factors, ascorbic acid, and the like. The catheter can also be used to deliver compounds to different regions of the anatomy in order to visualize the arteries and other blood vessels. Such compounds are well known to those of skill in the art and can include radio-opaque dyes and the like.

The catheter is selected from a variety of catheters that can perform procedures, for example, but not limited to, an ultrasound catheter, a suction catheter, a heat catheter, a mechanical catheter comprising a medical device, a laser catheter that may be used to break up clots in the carotids or the Circle of Willis, for example, and the like.

The drugs or other biologically active materials incorporated into the embolic protection device of the present invention perform a variety of functions. The functions include but are not limited to an anti-clotting or anti-platelet function; and preventing smooth muscle cell growth on the inner surface wall of the vessel. The drugs include but are not limited to drugs that inhibit or control the formation of thrombus or thrombolytics such as heparin or heparin fragments, aspirin, coumadin, tissue plasminogen activator (tPA), urokinase (uPA), hirudin, and streptokinase, antiproliferatives (methotrexate, cisplatin, fluorouracil, Adriamycin, and the like) antioxidants (ascorbic acid, carotene, B, vitamin E, and the like), antimetabolites, thromboxane inhibitors, non-steroidal and steroidal anti-inflammatory drugs, β-blockers, calcium channel blockers, genetic materials including DNA and RNA fragments, and complete expression genes, carbohydrates, and proteins including but not limited to antibodies (monoclonal and polyclonal) lymphokines and growth factors, prostaglandins, and leukotrienes. The stent also incorporates bioactive materials such as fibronectin, laminin, elastin, collagen, and intergrins. Fibronectin promotes adherence of the stent to the tissue of the vessel.

Clinical guidelines to ascertain how and if an individual is at risk for cerebrovascular embolism and/or recurrent stroke can be found in, for example, Albers (2004) “Antithrombotic and thrombolytic therapy for ischemic stroke: the Seventh ACCP Conference on Antithrombotic and Thrombolytic Therapy”, Chest, 126(3 Suppl): 483S-512S; Singer (2004) “Antithrombotic therapy in atrial fibrillation: the Seventh ACCP Conference on Antithrombotic and Thrombolytic Therapy”, Chest, 126(3 Suppl): 429S-456S; and Buller (2004) “Antithrombotic therapy for venous thromboembolic disease: the Seventh ACCP Conference on Antithrombotic and Thrombolytic Therapy”, Chest, 126(3 Suppl): 401S-428S.

The embolic protection device can be placed in any part of the systemic circulation to divert emboli away from unwanted locations or to less problematic locations. For example, in an individual having AF, the device can be i) placed in the aortic arch to deflect emboli towards the descending aorta; ii) placed at the bifurcation of common carotid artery, with the fin covering the ostium of the internal carotid artery in order to selectively divert emboli to external carotid artery. In another example the device is placed anywhere in the circulatory system at a bifurcation of two blood vessels but not the bifurcation of the common carotid artery into the external carotid artery and the internal carotid artery. In yet another example, the device can be placed in the abdominal aorta so that the fin may protect the ostia of the celiac trunk, the superior mesenteric artery, the inferior mesenteric artery, and hence divert emboli towards the femoral arteries. In a further example, for an individual with risk of deep venous thrombosis (DVT) embolizing towards the lung and having existing right to left shunts such as patent ductus arteriosus (PDA), ventricular septal defect (VSD), or atrial septal defect (ASD), the device or a plurality thereof can be placed in the pulmonary artery or other blood vessel(s) so as to divert emboli away from the lung and/or the brain.

The embolic protection device can also be used to treat aneurysms. The stent portion is placed downstream or upstream of an aneurysm with fin covering the aneurysm (see FIG. 11). In this case, the fin can comprise a material that is substantially impermeable and/or semi-permeable to molecules and cells in the blood, and for its circumferential span to cover any diameter of the aneurysm. Aneurysms can arise mostly on one side of the artery or blood vessel, but they can progressively dilate to weaken the whole circumference of the vessel. Therefore, it is advantageous if the device comprises a fin having boundaries that can attach to the wall of the blood vessel to reduce the effects of dilation. The boundaries of the fin can have attaching means, such as, but not limited to, a stylet, a hook, a clip, a staple, an adhesive, a coil, a barb, or the like.

The embolic protection device can be used in combination with any other surgical or clinical device or tool, such as, but not limited to, devices for sealing aneurysms, devices for pacing the heart, devices for ablating thrombi, devices for ablating tumors, devices for cauterizing tissues, devices for sensing pressures, devices for sensing flow rates, devices for sensing thermal changes, devices for detecting biological compounds, devices for detecting non-biological compounds, devices for detecting gases, devices for visual angiography, for angioplasty, for stenting, filters (for example, for treating DVT), or the like.

The embolic protection device can alternatively further comprise releasable attaching means (4), the attaching means being disposed at the junction of the fin and the stent portions of the device. The attaching means can be, for example, but not limited to, a lock, a stylet, a hook, a clip, a staple, an adhesive, a coil, a barb, or the like. The attaching means can be activated from a remote location using a variety of methods, such as, for example, but not limited to, a catheter comprising a device that can unlock the attaching means, a radio-activated device, a heat-activated device, a magnetic flied activated device, a liquid-activated device (such as, for example, a crystalline matrix that dissolves at a predetermined rate in body fluid), or the like. This device would enable the operator to remove the fin when it is not necessary in a further procedure or no longer necessary. This device can be used, for example, when the operator implants a stent in the aortic arch for whatever reason (such as treating atherosclerosis) and the embolic protection device is also necessary for use to also protect the organs in the upper body from embolism. In this case the operator does not need to insert an additional catheter and/or device for intraoperative embolic protection. Then once the procedure is complete, the fin is released and removed from the body of the individual allowing the stent to remain in place.

The embolic protection device can be manufactured so as to conform to an individual's own aorta, i.e. can be custom-molded. The shape and size of the system is determined using measurements taken from, for example, an electromagnetic scan of the patient's anatomy using imaging technology such as MRI, CAT scans, or the like.

List of Reference Numerals.
1. Embolic Protection Device
2. Stent
3. Fin
4. Attaching Means
5. Fine Wires
6. Cross-wires
7. Catheter
8. Balloon or Cuff
9. Subclavian Artery
10. Common Carotid Artery
11. Brachiocephalic Artery
12. Internal Carotid Artery
13. Vertebral Artery
14. Basilar Artery
15. Posterior Cerebral Artery
16. Middle Cerebral Artery
17. Anterior Cerebral Artery
18. Aortic Arch
19. Emboli
20. Walls of Aorta
21. Blood Vessels
22. Synthetic Tubing (Aortic Arch)
23. Synthetic Tubing (Arterial Vessels)
24. Distal End of Catheter
25. Aperture for Catheter
26. Blood Vessel Lumen
27. Blood Vessel Wall
28. Aneurysm
29. Celiac Artery
30. Superior Mesenteric Artery
31. Renal Artery
32. Abdominal Aorta
33. Inferior Mesenteric Artery
34. Left Common Iliac Artery
35. Right Common Iliac Artery
36. Internal Iliac Artery
37. External Iliac Artery
38. Femoral Artery

The invention will be more readily understood by reference to the following examples, which are included merely for purposes of illustration of certain aspects and embodiments of the present invention and not as limitations.

### EXAMPLES

### Example I: Deploying the Embolic Protection Device

The following experiments aimed to answer the following two questions.
1. Can a device with a stent-like base and an extending diverter fin, placed in a model of the aortic arch divert simulated emboli?
2. In the same model of the aortic arch, is it possible to gain catheter access to the brachiocephalic trunk, left common carotid, and left subclavian arteries with such a device in place?

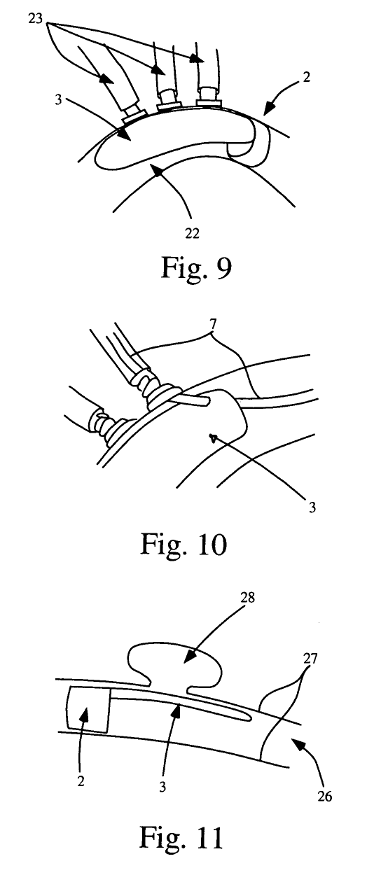

A simulated aortic arch was constructed using TYGON tubing comprising an “aortic arch” (22) and the “three arterial vessels” (23). Flexible tubing was used that could easily be bent to simulate the aortic arch, and then valves and tubing were connected to simulate the three arteries branching off of the aortic arch. The embolic protection device was placed in the lumen of the “aortic arch” (22). (See FIG. 9.)

Mustard seeds were used to simulate emboli, and the tubing system was connected to a heart simulator (Harvard Apparatus Pulsatile Blood Pump, Harvard Apparatus, Holliston Mass.). The heart simulator pumped water through the system in a regulated manner simulating the rhythm and pressure of the heart. The settings were as follows:
Output Phase Ratio: % systole/% diastole=40/60
Rate Pump (RPM): ˜40
CC/stroke: ˜28
   Results

1. The results of introducing emboli (simulated using mustard seeds) in a stream (water substituted for blood) flowing through the aortic arch with and without the device in place were compared. In order to prove efficacious the device would need to divert 100% of the emboli downstream, away from the brain. With the device placed in the model aortic arch (shown in FIG. 8, it was found that all emboli were diverted downstream (away from the “three arterial vessels”), where they would cause less damage in vivo.

2. The device was placed within the simulated aortic arch with the diverting fin extending across the “three arterial vessels”. With this construction in place a catheter was fed through the distal end in an attempt to gain access to the three arteries. Given the flexible nature of the diverting fin, access was gained to the three major arteries stemming from the aortic arch (shown in FIG. 10).

All the mustard seeds (simulated emboli) were diverted from the openings to the simulated three arteries.

Those skilled in the art will appreciate that various adaptations and modifications of the just-described embodiments can be configured without departing from the scope and spirit of the invention. Other suitable techniques and methods known in the art can be applied in numerous specific modalities by one skilled in the art and in light of the description of the present invention described herein. Therefore, it is to be understood that the invention can be practiced other than as specifically described herein. The above description is intended to be illustrative, and not restrictive. Many other embodiments will be apparent to those of skill in the art upon reviewing the above description. The scope of the invention should, therefore, be determined with reference to the appended claims, along with the full scope of equivalents to which such claims are entitled.

1. An embolic protection device comprising a body and a fin, the body adapted for placement within the lumen of a blood vessel and the fin adapted for placement against a wall of the blood vessel and an opening of at least one other blood vessel that arises from the blood vessel.

2. The embolic protection device of claim 1 wherein the blood vessels are selected from the group consisting of the aorta, the brachiocephalic artery, the common carotid artery, the subclavian artery, the thoracic intercostal arteries, the celiac artery, the superior mesenteric artery, the renal arteries, the inferior mesenteric artery, the left common iliac artery, the right common iliac artery, the internal iliac arteries, the external iliac arteries, the femoral arteries, the pulmonary artery, and the heart chamber.

3. The embolic protection device of claim 1 wherein the fin having apertures sized to obstruct selectively the passage of particulate emboli.

4. The embolic protection device of claim 3, wherein the apertures are sized to obstruct particulate emboli, the particulate emboli being at least 100 μm in size.

5. The embolic protection device of claim 4, wherein the apertures are sized to obstruct particulate emboli, the particulate emboli being at least 200 μm in size.

6. The embolic protection device of claim 5, wherein the apertures are sized to obstruct particulate emboli, the emboli being at least 300 μm in size.

7. The embolic protection device of claim 1, further comprising a deploying catheter.

8. The embolic protection device of claim 1, further comprising a sensor catheter.

9. The embolic protection device of claim 1, wherein the body is a stent.

10. The embolic protection device of claim 1 wherein the fin is longer than the body.

11. The embolic protection device of claim 1 further comprising a drug-eluting composition.

12. The embolic protection device of claim 1 further comprising releasable attaching means.

13. An embolic protection device comprising a body and a fin, the body adapted for placement within the lumen of the aorta and the fin adapted for placement against the superior wall of the aortic arch and the opening of blood vessels that arise from the superior portion of the aortic arch.

14. A method of using an embolic protection device to prevent a cerebrovascular embolism in an individual, the method comprising the steps of
i) providing an individual at risk for having a cerebrovascular embolism,
ii) providing the embolic protection device of claim 1 and a deploying catheter comprising a lumen, the stent and fin being foldedly disposed in the lumen of the deploying catheter,
iii) inserting a catheter having a lumen into a blood vessel of the individual,
iv) advancing the distal end of the catheter to a desired position in the blood vessel, the desired position being proximal to an opening of another blood vessel,
v) inserting a guide-wire through the lumen of the catheter,
vi) advancing the guide-wire so that the distal end of the guide-wire is positioned proximal to the distal end of the catheter,
vii) withdrawing the catheter,
viii) advancing the deploying catheter comprising the folded embolic protection device over the guide-wire so that the distal end of the deploying catheter is proximal to the distal end of the guide-wire,
ix) deploying the stent and fin from the deploying catheter so that the stent is opened and positioned against the wall of the blood vessel and the fin is positioned against the opening of the other blood vessel,
x) withdrawing the catheter from the blood vessel, and
xi) leaving the stent and fin in place for a desired time period thereby preventing a cerebrovascular embolism.

15. A method of using an embolic protection device to treat an individual having had a stroke to prevent recurrent stroke the method comprising the steps of
i) providing an individual having had a stroke,
ii) providing the embolic protection device of claim 1 and a deploying catheter comprising a lumen, the stent and fin being foldedly disposed in the lumen of the deploying catheter,
iii) inserting a catheter having a lumen into a blood vessel of the individual,
iv) advancing the distal end of the catheter to a desired position in the blood vessel, the desired position being proximal to an opening of another blood vessel,
v) inserting a guide-wire through the lumen of the catheter,
vi) advancing the guide-wire so that the distal end of the guide-wire is positioned proximal to the distal end of the catheter,
vii) withdrawing the catheter,
viii) advancing the deploying catheter comprising the folded embolic protection device over the guide-wire so that the distal end of the deploying catheter is proximal to the distal end of the guide-wire,
ix) deploying the stent and fin from the deploying catheter so that the stent is opened and positioned against the wall of the blood vessel and the fin is positioned against the opening of the other blood vessel,
x) withdrawing the catheter from the blood vessel, and
xi) leaving the stent and fin in place for a desired time period thereby preventing recurrent stroke.

16. A method of using an embolic protection device to treat an individual having an aneurysm the method comprising the steps of
i) providing an individual having an aneurysm,
ii) providing the embolic protection device of claim 1 and a deploying catheter comprising a lumen, the stent and fin being foldedly disposed in the lumen of the deploying catheter,
iii) inserting a catheter having a lumen into a blood vessel of the individual,
iv) advancing the distal end of the catheter to a desired position in the blood vessel, the desired position being proximal to the aneurysm,
v) inserting a guide-wire through the lumen of the catheter,
vi) advancing the guide-wire so that the distal end of the guide-wire is positioned proximal to the distal end of the catheter,
vii) withdrawing the catheter,
viii) advancing the deploying catheter comprising the folded embolic protection device over the guide-wire so that the distal end of the deploying catheter is proximal to the distal end of the guide-wire,
ix) deploying the stent and fin from the deploying catheter so that the stent is opened and positioned against the wall of the blood vessel and the fin is positioned against the aneurysm,
x) withdrawing the catheter from the blood vessel, and
xi) leaving the stent and fin in place for a desired time period thereby treating the individual having the aneurysm.

17. A method of using an embolic protection device to prevent a pulmonary embolism in an individual, the method comprising the steps of
i) providing an individual at risk for having a pulmonary embolism,
ii) providing the embolic protection device of claim 1 and a deploying catheter comprising a lumen, the stent and fin being foldedly disposed in the lumen of the deploying catheter,
iii) inserting a catheter having a lumen into a blood vessel of the individual,
iv) advancing the distal end of the catheter to a desired position in the blood vessel, the desired position being proximal to an opening of another blood vessel,
v) inserting a guide-wire through the lumen of the catheter,
vi) advancing the guide-wire so that the distal end of the guide-wire is positioned proximal to the distal end of the catheter,
vii) withdrawing the catheter,
viii) advancing the deploying catheter comprising the folded embolic protection device over the guide-wire so that the distal end of the deploying catheter is proximal to the distal end of the guide-wire,
ix) deploying the stent and fin from the deploying catheter so that the stent is opened and positioned against the wall of the blood vessel and the fin is positioned against the opening of the other blood vessel,
x) withdrawing the catheter from the blood vessel, and
xi) leaving the stent and fin in place for a desired time period thereby preventing a pulmonary embolism.
